(19) United States
(12) Patent Application Publication
(10) Pub. No.: US 2026/0157739 A1
(43) Pub. Date: Jun. 11, 2026

(54) A RETRACTION DEVICE WITH ARRANGEMENT OF HOOK, CLAMP AND ELASTIC MEMBER

(71) Applicants: Mahesh Ramola, Dehradun (IN); Monika Ramola

(72) Inventor: Mahesh Ramola, Dehradun (IN)

(21) Appl. No.: 18/708,502

(22) PCT Filed: Apr. 18, 2024
(86) PCT No.: PCT/IB2024/053783

§ 371 Date: May 8, 2024

(30) Foreign Application Priority Data
Jul. 28, 2023 (IN) 202311051081

(51) Int. Cl. A61B 17/02; A61B 17/00

(52) U.S. Cl. CPC A61B 17/0206; A61B 2017/0046; A61B 2017/0287

(57) ABSTRACT

A retraction device 100 for retracting one or more muscles or skin flap of a patient during surgery is provided. The device includes a first plate 1 and second plate 8 adapted to connect with a fixed structure. The first plate 1 having a plurality of detachable hooks 9 mounted thereon such that at least one detachable hook selected from the plurality of detachable hooks 9 is connected to a first end of one or more elastic members 14. Further, a second end of the one or more elastic members is connected to the one or more muscles/skin flap 15 to be retracted through a muscle connecter means 16. The one or more elastic members are adapted to retract the one or more muscles or skin flap of the patient during the surgery

### TECHNICAL FIELD

The present disclosure relates to a skin and muscle retractor device, and more particularly relates to retraction device with hook, clamp and elastic member arrangement for enabling surgeons to effectively retract muscles of a patient during a surgery (such as but not limited to brain and spine surgery).

### BACKGROUND

A retractor is a tool that is used to manipulate the tissues of the body to make the surgical procedure easier to perform. The broad term retractor typically describes a simple steel metal possessing a straight curved, hooked, or angled blade/tube/prongs, which is manually manipulated to help maintain a desired position of tissue during surgery. More sophisticated retractors may be clamped in place (usually to a tableside frame) or suspended at the end of a robotic arm. Retractors can also be “self-retaining” and no longer need to be held once inserted, having two or more opposing blades or hooks which are separated via spring, ratchet or other method. The term retractor is also used to describe distinct, hand-cranked devices such as rib spreaders (also known as thoracic retractors, or distractors) with which surgeons may use to forcefully drive tissues apart to obtain exposure. Different surgery specialties can have specific kinds of retractors-e.g., for certain kinds of spinal surgery, such as minimally invasive transforaminal lumbar interbody fusions, some retractors are fitted both with suction and with fibreoptic lights to keep deep surgical wounds both dry and illuminated.

The retractor is commonly used in surgeries that involve exposing/retraction of the organs. When an organ is located beneath another organ or muscles, the retractor can be used to adjust the tissues so the surgeon can easily see and access the organ.

For example, temporalis muscle (TM) retraction and skin flap retraction is a common pathway on access to many brain surgeries. Traditionally, most common instrument used for TM retraction is Yasargil spring hook retractor (SHR) which has hooks, spring mechanism and bulldog clamp/attachments as its essential components. Following are some major limitations of existing temporalis muscle and skin flap retraction instruments:

A most important concern is that on repeated use of the SHR a frequent wear and tear of spring mechanisms of SHR is a common occurrence. It causes loss of elasticity of spring, loosening, breakage of metal wire and decrease grip of bulldog clamp leading to impaired retraction and need for frequent repair and replacement. Also, while the fastening element (bulldog clamp) is fastened to sterile surgical drape, with the force of retraction and tension of spring, fastening element may slip on sterile coverings, and thus requirement of repeated corrections in fastening clamp may compromise sterile environment. Moreover, fine hooks of SHR are sharp and used for holding and grasping tissue and skin. The hook end is by design made sharp to increase the grip on soft tissue. During fixing and manipulation of hook, its sharp end may cause finger injury to the surgical team.

Similarly, a paravertebral musculature (PVM) retraction is a common surgical step to access deep seated neurological structures in thorax and lumbar spine surgery. Many open and minimal invasive retraction system are used for PVM retraction. The purpose of PVM retraction in spine surgery is to provide direct visualization of deep-seated spine pathology and provide adequate working space for surgeon with minimum possible retraction induced damage to surrounding tissue. Example of common spine retractor system is Caspar microlumbar discectomy retractor system (Aesculap implant system). Following are some major limitations of existing spine retractor instruments:

Many of the spine retractors used during PVM retraction occupy surgical space as these retractors have metal blades or tubes or prongs which reduces available working surgical space. Also, the metal blade/tube/prongs of retractors, used during PVM retraction, under tension for long periods may cause retraction induced injury. Further, since many of the available retractors are made of metals which are radiopaque and causes significant light reflection. Radiopaque nature of commonly used metals interferes with intraoperative fluoroscopy and operation light falling on metal retractors causes glare and may cause temporary blindness to surgeon during surgery. Though using radiolucent metal in manufacturing and black coating on retractor blades addresses these shortcomings but comes at significantly increased cost of equipment. It is further concerning that, depending upon patient weight and body habitus, conventional spine retractors require multiple sets of instruments of different sizes to effectively retract PVM for optimal fit and avoid increasing distance from surgical target. Also, another common problem is frequent drifting of blades/tube of spine retractor which leads to inconsistent and inadequate retraction.

Therefore, there is a need for an improved retraction device that overcomes the limitations of conventional techniques, provides secure, stable, and reliable retraction during surgery, and also enhances the overall efficiency of surgical procedures.

### SUMMARY

The present invention is directed to provide a retraction device with clamp, hook and elastic member arrangement for enabling surgeons to effectively retract muscles of a patient during a surgery.

More particularly, the present invention is directed to provide a retraction device with clamp, hook and elastic member arrangement, preferably for brain and spine surgery but not limited merely therefore, capable of retracting muscles/tissues around brain and spine with a convenient coupling procedure and securing a surgical space and view by proper grip of skin and/or muscle during brain and/or spine surgery.

According to an aspect of the present invention, there is provided a retraction device. The device has following essential parts/components: clamping plates, a plurality of detachable hooks, and at least one elastic member (elastic band).

A first end of the at least one elastic member (elastic band) is connected to at least one detachable hook of a plurality of detachable hooks and a second end of the at least one elastic member (elastic band) is connected to a muscle connecter means (say for example a surgical suture or a small hook). In an aspect, first muscle connector is applied then elastic member is attached to hook.

In another aspect, the muscle connecter means is operatively connected to portion of a muscle that needs retraction. It may be appreciated that, even though the term “muscle connecter means” is used, it do not necessarily should be considered as limiting or restricting to be applicable only for muscles. The muscle connecter means can also be connected to portion of skin that needs retraction.

Thus, the elastic member (elastic band) actively retracts soft tissue, muscle, skin and remains in surgical field and the hook provides attachment to the elastic member (elastic band) and remains passively attached to any of the stable stationary surgical paraphernalia (SSP) like edge of mayo trolley, siderail of operation table or right-angle bar etc.

### BRIEF DESCRIPTION OF DRAWINGS

The detailed description is described with reference to the accompanying figures. In the figures, the left-most digit(s) of a reference number identifies the figure in which the reference number first appears. The same numbers are used throughout the drawings to refer like features and components.

FIG. 1 illustrates a top view of the retraction device in accordance with the exemplary embodiment of the present subject matter.

FIG. 2 illustrates a clamping side view, of the retraction device in accordance with the exemplary embodiment of the present subject matter.

FIG. 3 illustrates a bottom view of the retraction device in accordance with the exemplary embodiment of the present subject matter.

FIG. 4 illustrates a side view, of the retraction device in accordance with the exemplary embodiment of the present subject matter.

### DETAILED DESCRIPTION

Some embodiments of the present disclosure, illustrating all its features, will now be discussed in detail. It must also be noted that as used herein and in the appended claims, the singular forms “a”, “an” and “the” include plural references unless the context clearly dictates otherwise. The present disclosure, in an exemplary embodiment discloses an improved retraction device for medical surgeries.

### PART LIST

Retraction device 100
First plate 1
First surface 2
Plurality of holes 3
Pin Joint 4
Second edge 5
Base plate 6
Second surface 7
Second plate 8
Plurality of hooks 9
Clamping screw 11
Elastic member/s 14
Muscles/Skin flap nearby Surgical site 15
Muscle connecter means 16

Embodiments of the present invention is directed to provide a retraction device with clamp, hook and elastic member arrangement, preferably for brain and spine surgery but not limited merely therefore, capable of retracting muscles/tissues around brain and spine with a convenient coupling procedure and securing a surgical space and view by proper grip of skin and/or muscle during brain and/or spine surgery.

According to an embodiment of the present invention, a retraction device is provided. The device has following essential parts/components: clamping plates, a plurality of detachable hooks, and at least one elastic member (elastic band).

A first end of the at least one elastic member (elastic band) is connected to at least one detachable hook of a plurality of detachable hooks and a second end of the at least one elastic member (elastic band) is connected to a muscle connecter means (say for example a surgical suture or a small hook).

In an embodiment, first muscle connector is applied then elastic member is attached to hook.

In another embodiment, the muscle connecter means is operatively connected to portion of a muscle that needs retraction. It may be appreciated that, even though the term “muscle connecter means” is used it do not necessarily should be considered as limiting or restricting to be applicable only for muscles. The muscle connecter means can also be connected to portion of skin that needs retraction.

Thus, the elastic member (elastic band) actively retracts soft tissue, muscle, skin and remains in surgical field and the hook provides attachment to the elastic member (elastic band) and remains passively attached to any of the stable stationary surgical paraphernalia like edge of mayo trolley, siderail of operation table or right-angle bar etc.

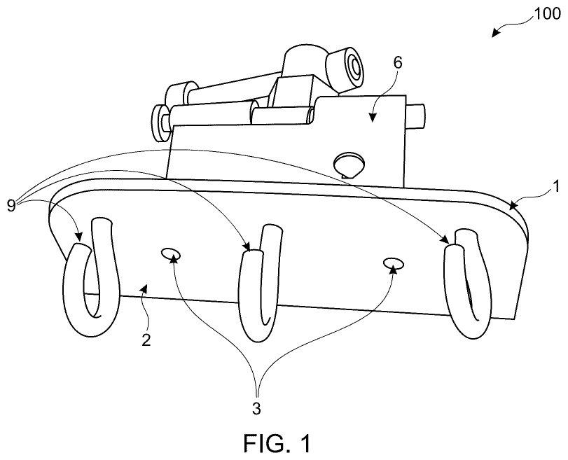

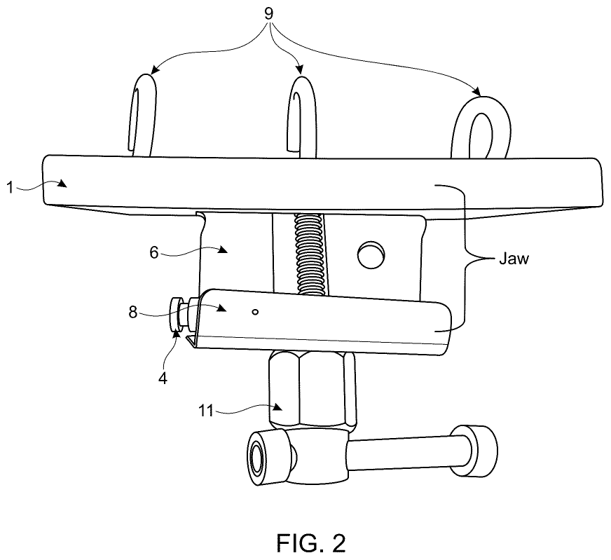

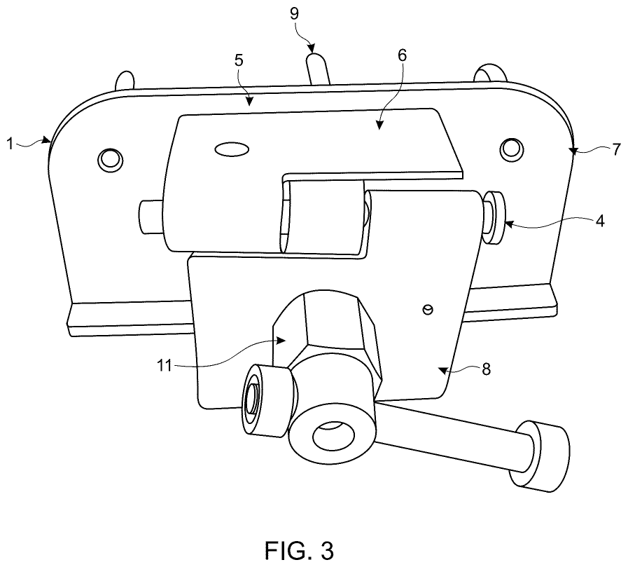

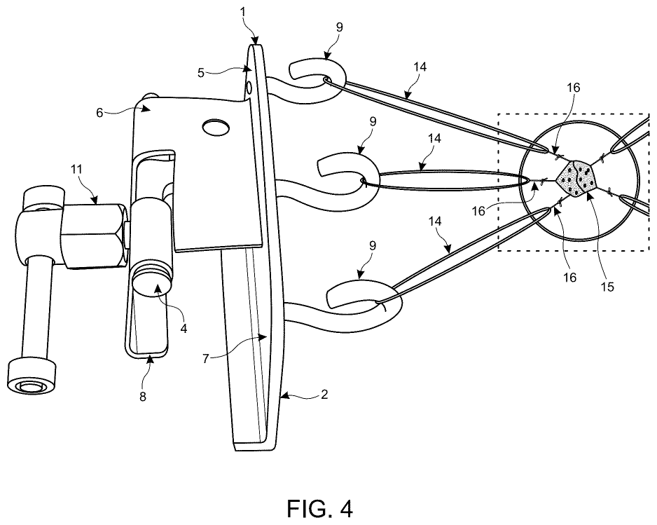

FIG. 1 illustrates a top view of the retraction device in accordance with the exemplary embodiment of the present subject matter. FIG. 2 illustrates a clamping side view of the retraction device in accordance with the exemplary embodiment of the present subject matter. FIG. 3 illustrates a bottom view of the retraction device in accordance with the exemplary embodiment of the present subject matter. FIG. 4 illustrates a side view, of the retraction device in accordance with the exemplary embodiment of the present subject matter

Now, to elaborate on FIGS. 1-4, the embodiment discloses an exemplary retraction device (100). The retraction device (100) may comprise of a disposable component (use and throw), and a reusable component (re-usable). The disposable component may comprise of elastic member (14) like rubber-bands or elastic bands. Further the elastic members (14) (rubber-band or the elastic bands) may be used to retract muscle (15), soft tissues and skin flap (15). The elastic member may be configured to use their elastic properties mimicking a spring. Further the elastic member may be suture with anchors on a first end to hold the muscle/skin/soft tissue and may be removably placed on a hook at a second end.

The hooks (9) may be removable mounted on the reusable component. The reusable component remains passively attached to any of the stable surgical paraphernalia like edge of mayo trolley, siderail of operation table or right-angle bar etc. Further the reusable component may comprise a pair of clamping plates (i.e., first plate (1) and second plate (8)) forming jaw (U-sharped structure) and a clamping screw (11).

The reusable component may comprise a plurality of hooks (9) removable mounted on a first plate (1). The first plate (1) may be a part of the pair of jaws, and is further provided with a plurality of holes (3). The plurality of holes is provided on one of a surface (viz., first surface (2)) of the first plate (1). Further the plurality of holes (3), are configured to receive the plurality of hooks (9). Further the plurality of hooks (9) is removably mounted in the plurality of holes (3). Further, position and pattern of the plurality of hooks (9) on the first plate (1) may be changeable, thus enabling the retraction device (100) to be adapted for all patients irrespective of weight and size of patients.

The first plate (1) serves dual function-it acts as base for attachment of hooks (9) as well as forms an upper jaw of jaw (U-sharped structure) of the instrument. The plurality of hooks (9) has threads upon its exterior which enters the corresponding grooves upon the inside of holes present in first plate (1). The number, direction and position of hooks (9) on the first plate (1) can be changed as per surgeon's preference.

The first plate (1) forming upper jaw of retraction device (100) may be positioned horizontal and may have dimension about 10 cm×5 cm in size. The first plate (1) may further have a downcurve 90 degrees at its clamping end in accordance an aspect of the first plate. This clamping end acts as upper jaw and fits into stable surgical paraphernalia. Further a lower plate (second plate (8)) of size approximately 4.8 cm×3.5 cm is present below which is also upcurved 90 degrees at its clamping end. The second plate (8) serves two functions—it forms lower jaw for fitting into stable surgical paraphernalia and also gives attachment to the clamping screw (11) at its inferior surface.

The clamping screw (11) also serves dual function by regulating jaw opening (i.e., distance between the first pate (1) and the second pate (8)) as well as locking the retraction device to the stable surgical paraphernalia. The jaw opening is customizable and can be increased or decreased as per surgeon's choice and available operating room setup.

In accordance with another exemplary embodiment a retraction device (100) is disclosed. The retraction device may comprise a first plate (1). The first plate (1) may comprise a first surface (2) and a second surface (7). The first surface (2) and the second surface (7) define the first plate (1).

The first plate (1) may further have a plurality of holes (3) distributed in a defined pattern over the first surface (2) of the first plate (1). The plurality of the holes (3) may be provided with internal threads. The plurality of holes (3) may be configured to receive a plurality of hooks (9). Further, the plurality of hooks (9) may be fastened in the plurality of holes (3), by thread engagement of the threads provided on the plurality of holes (3) and the plurality of hooks (9). The plurality of hooks (9) is removably fastened on the first plate (1), and further the position of the hooks (9) may be changed on the first plate (1), by engaging with the plurality of holes (3) provided/distributed in a pattern on the first surface (2) of the first plate (1).

In accordance with the exemplary embodiment, the plurality of hooks (9) may be configured to engage with elastic member/s (14). Further, the elastic member (14) may be simultaneously engaged with plurality of muscles connector means (16). Further, the plurality of muscles connector means (16) may be inserted in muscle/skin/soft tissues in nearby surgical site (15)) to retract/open/pull apart the muscle, skin, soft tissue so as to enable the surgeon to perform surgery.

Further, the first plate (1) has a base plate (6) that may be mounted on the first plate (1) and adjacent to the second edge (5). The base plate (6) extends away from the second surface (7) of the first plate (1) to connect with a second plate (8) of the retraction device (100).

The second plate (8) is movably mounted on the base plate (6). The second plate (8) is movably connectable to the second edge (5). Upon displacement, which is achieved by means of a clamping screw (11), the second pate (8) may move towards or away from the first plate (1), and it can reduce or increase the distance the first and second plate. Reducing distance between two plates increases grip on stable surgical paraphernalia, thereby acting as jaw to hold firmly on stable surgical paraphernalia. Increasing distance by means of clamping screw decreases grip on stable surgical paraphernalia and device can be unmounted. Further, a pin joint is placed between the second plate (8) and the base plate (6) to enable moveable connection. The clamping screw (11) is mounted between the first plate (1) and the second plate (8).

Specifically, referring to FIG. 1, it illustrates a top view of the retraction device in accordance with the exemplary embodiment of the present subject matter. The retraction device 100, may comprise a first plate 1. The first plate 1 comprises a first surface 2. The first surface 2, of the first plate may comprise a plurality of holes 3. The plurality of holes 3 may be distributed across the first surface 2 in a defined pattern. The first plate 1 may further comprise a plurality of hooks 9 fastened in the plurality of holes 3. The fastening between the plurality of hooks 9 and the plurality of holes 3 may be via screw-threads.

Specifically, referring to FIG. 2, it illustrates a clamping side view, of the retraction device 100 in accordance with the exemplary embodiment of the present subject matter. In accordance with the aspect of the exemplary embodiment, the first plate (1) has a base plate (6) that may be mounted on the first plate (1). The base plate (6) extends away from the first plate (1) to connect with a second plate (8) of the retraction device (100). The second plate (8) may be movably mounted on the base plate (6). Upon displacement, which is achieved by means of a clamping screw (11), the second pate (8) may move towards or away from the first plate (1), and it can reduce or increase the distance the first and second plate. Reducing distance between two plates increases grip on stable surgical paraphernalia, thereby acting as jaw to hold firmly on stable surgical paraphernalia. Increasing distance by means of clamping screw decreases grip on stable surgical paraphernalia and device can be unmounted.

Further, a pin joint (4) may be placed between the second plate (8) and the base plate (6) to enable moveable connection. The clamping screw (11) is mounted between the first plate (1) and the second plate (8).

Further the clamping screw 11 may be connected to a handle to enable tightening or loosening of the pair of the jaw. The tightening or loosening enables the first plate 1 and the second plate 8 to move towards each other or away from each other based on the direction of the rotation of the clamping screw 11 by the handle.

Specifically, referring to FIG. 3, it illustrates a bottom view of the retraction device 100 in accordance with the exemplary embodiment of the present subject matter. The base plate 6 mounted on the first plate 1. The base plate (6) is mounted adjacent to a second edge 5 of a second surface 7 of the first plate 1. Further the second plate (8) is connected to the base plate (6). The second plate (8) may be curved to form a lower jaw for the retraction device 100.

Further the base plate 6 is mounted on the first plate 1. The base plate 6 may be coupled with the second plate 8. The second plate 8 may be moveable with respect to the base plate 6. Further the pin joint (4) may be placed between the second plate 8 and the base plate 6 enabling the second plate 8 to move a defined distance.

In a preferred embodiment, FIG. 4 illustrates a side view, of the retraction device in accordance with the exemplary embodiment of the present subject matter.

A retraction device 100 for retracting one or more muscles or skin flap of a patient during surgery is provided. The device includes a first plate 1 adapted to connect with a fixed structure. The first plate 1 having a plurality of detachable hooks 9 mounted thereon such that at least one detachable hook selected from the plurality of detachable hooks 9 is connected to a first end of one or more elastic members 14. Further, a second end of the one or more elastic members is connected to the one or more muscles or skin flap 15 to be retracted through a muscle connecter means 16. The one or more elastic members are adapted to retract the one or more muscles or skin flap of the patient during the surgery.

In an implementation, the rubber band used as elastic members for retraction are inexpensive and single use disposable item. Usually, 5-8 rubber bands are used in a case and cost of a single pack of 10 rubber bands is Indian rupee (INR) 1 or US $ 0.012, per case. In other words, 1 US dollar is enough for 85 surgeries. Presently the making cost of retraction device device is around 5000 INR or 60 US dollar.

Certain advantages of using elastic members instead of spring or any other mechanisms for retracting of muscles while surgery are provided below:

It may be appreciated that, since elastic members have no sharp surface, the risk of accidental finger injury during its use is zero.

It may be appreciated that, revisions of elastic member attachment during surgery does not risk any breach in sterile environment.

If any elastic member malfunctions, replacement is easy due to plentiful availability owing to its very low cost. Moreover, elastic member retraction provides more flatter field, less metal and spring in the surgical vicinity.

In spine surgery, conventional spine retractors consist of metal blades or tubes/prongs which occupies space in surgical wound, thereby reducing the working space for surgical manipulations. Retraction by elastic members takes negligible space and almost all of the surgical wound cavity is available for surgeon to work.

With retraction device, one size fits all. same system can be used for all patients irrespective of weight and size of patients.

Operating room lights or microscope lights gets reflected from metal blades of conventional retractor which can temporarily blind the surgeon. Manufacturers need to add black coating to instruments to make them less reflective but this increases the cost of instrument. Elastic members do not reflect light, so there is no risk of light reflection causing temporary surgeon blindness.

Intraoperative fluoroscopy is required in spine surgery to confirm correct location of surgery. Most routinely used metal retractors are radiopaque which causes interference in intraoperative fluoroscopy during surgery. Manufacturers needs to use special metal alloy to make retractors radiolucent, but this adds to the cost of instrument. Elastic members are radiolucent and does not interfere in intraoperative images.

The other limitations of current spine retractors is that sometimes despite maximal tightening, retractor blades/tubes/prongs continue to drift, which makes unstable and inefficient retraction. There is also risk of retraction induced injury due to hard, inflexible metal blades retracting tissues for long periods during surgery.

The retraction provided by elastic members in retraction device does not drift and surgeon gets consistent and stable retraction of PVM. The risk of retraction induced injury is minimal due to soft consistency of elastic members.

The retraction device is effective and maintains longer period of consistent retraction without retraction induced injury.

It does not encroach available surgical space, provides flatter field and facilitates exposure of deeper structures.

Because of less obstructive structure and composition compared to conventional retractors, the retraction device allows smoother and easier manipulations especially in the deeper operative fields.

The innovative aspect of new retractor system includes having fewer parts and simple design, which makes it durable and less prone to routine wear, tear.

It is safe, effective, economical, easy to use and has short learning curve with low maintenance cost.

In an exemplary embodiment, the first plate 1 is a clamping plate.

In an exemplary embodiment, the fixed structure is selected from an edge of a trolley used in operating room, a siderail of an operation table, or a right-angle bar of the operation table.

In an exemplary embodiment, the clamping plate is operatively coupled to the fixed structure using one or more clamping screw 11 provided on the retraction device 100.

In an exemplary embodiment, the muscle connecter means 16 is selected from any or a combination of a surgical suture taken on the one or more muscles or skin flap to be retracted.

In an exemplary embodiment, the elastic members may include a member or a part having elasticity property that is formed of polymeric materials. The elastic members may be selected from a rubber band, latex, Lycra and nylon..

In an exemplary embodiment, the one or more elastic members is an elastic thread.

Thus, it can be noted form the above that the retraction device consists of two components-disposable and Reusable. Disposable component consists of elastic members which are used to retract muscle and/or skin flap. This portion of retraction device actively retracts muscle, skin, soft tissue and remains in surgical field. Reusable component is made of stainless steel. It provides attachment to elastic members and remains passively attached to any of the stable surgical paraphernalia like edge of mayo trolley, siderail of operation table or right-angle bar etc. Retraction device has 3 parts - hooks, jaws, and clamping screw.

Hooks 9 are about 1.6 cm high question mark shaped structure which receives and engages elastic members. Hooks are detachable which can be attached to any of the multiple holes on the first plate 1. First plate 1 serves dual function-it acts as base for attachment of hooks as well as forms the upper jaw of the instrument. Hooks have threads upon its exterior which enters the corresponding grooves upon the inside of holes present in first plate. The number, direction and position of hooks on first plate can be changed as per surgeon's preference.

First plate 1 forming upper jaw of retraction device is about 10 cm×5 cm in size. It has multiple holes in it which have grooves inside to receive the threaded exterior of hook. It is downcurved 90 degrees at its clamping end. This clamping end acts as upper jaw and fits into stable surgical paraphernalia. Another plate 8 of size approximately 4.8 cm×3.5 cm is present below which is also upcurved 90 degrees at its clamping end. Second plate serves two functions—it forms lower jaw for fitting into stable surgical paraphernalia and also gives attachment to the clamping screw at its inferior surface.

Clamping screw 11 also serves dual function. it regulates jaw opening as well as locks retraction device to the stable surgical paraphernalia. The range of jaw opening is 0.5 to 3 cm which fits in our neurosurgery operation room. However, jaw opening is customizable and can be increased or decreased as per surgeon's choice and available operating room setup. To close jaws turn clamping screw clockwise and to open jaws turn clamping screw anticlockwise.

### WORKING OF THE INVENTION AND IMPLEMENTATION DETAILS

### Best Mode

Use of retraction device in craniotomy: Following skin incision in anterior or anterolateral craniotomy, anterior retraction of skin flap together with dissected temporalis muscle is required to access deeper structures. Simple surgical suture tied to elastic member is taken at the base of dissected temporalis muscle as low as possible. elastic members are now stretched and attached to hook present on first plate which is clamped to stable surgical paraphernalia. This keeps constant and stable retraction on the dissected temporalis muscle. Similar elastic member retraction is placed on dissected skin flap also. Usually, 5 to 8 elastic members with simple suture are needed. The number, direction, angle and tension of each elastic members can be adjusted as per surgeon's choice by changing the orientation and placement of hook on the first plate of retraction device.

Use of retraction device in lumbar spine surgery: The technique is similar as described above. In lumbar spine surgery silk suture tied to elastic members are passed through the bulky paravertebral muscle. Elastic members are then attached to hook on the first plate of retraction device which is clamped to stable surgical paraphernalia. This type of retraction is safe and effective in both open and minimal invasive spine surgery. The number, direction, angle and tension of elastic member retractions can be changed as per requirement by changing direction and placement of hook on first plate. Depending on the patient's habitus and surgeon's preference paravertebral muscle retraction can be done with elastic member alone or combined with a small handheld longneck retractor. After the neurosurgical procedure, elastic members could be easily and quickly removed from the surgical field in both craniotomy and lumbar spine surgery.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

1. A retraction device 100 for retracting one or more muscles or skin flap of a patient during surgery, the device characterized in that having:
a first plate 1 adapted to connect with a fixed structure, wherein the first plate 1 having a plurality of detachable hooks 9 mounted thereon; and
one or more elastic members 14 wherein a first end of one or more elastic members 14 connected to at least one detachable hook selected from the plurality of detachable hooks 9, and
wherein a second end of the one or more elastic members is connected to the one or more muscles/skin flap 15 to be retracted through a muscle connecter means 16; and
wherein the one or more elastic members are adapted to retract the one or more muscles/skin flap/soft tissues of the patient during the surgery.

2. The retraction device 100 as claimed in claim 1, wherein the first plate 1 is a clamping plate, and the fixed structure selected from an edge of a trolley used in operating room, a siderail of an operation table, or a right-angle bar of the operation table, and wherein the clamping plate is operatively coupled to the fixed structure using one or more clamping screw 11 of the retraction device 100.

3. The retraction device 100 as claimed in claim 1, wherein the muscle connecter means 16 is selected from any or a combination of a surgical suture taken on the one or more muscles/skin flap/soft tissue to be retracted or one or more small hooks.

4. The retraction device 100 as claimed in claim 1, wherein the one or more elastic members are selected from a rubber band, latex, lycra, and nylon.

5. The retraction device 100 as claimed in claim 1, wherein the one or more elastic members is an elastic thread.

6. The retraction device 100 as claimed in claim 1, wherein the first plate 1 comprises a first surface 2 and a second surface 7;
a plurality of holes 3 on the first surface 2 of the first plate 1, wherein each of the plurality of holes 3 is adapted to receive threaded exterior end of the at least one detachable hook selected from the plurality of detachable hooks 9;
a second edge 5 on the first plate 1;
a base plate 6, mounted on the first plate 1 and adjacent to the second edge 5, wherein the base plate 6 extends away from the second surface 7 on the first plate 1; and
a second plate 8, movably mounted on the base plate 6, wherein the second plate 8 is movably connected at a distal end from the second edge 7.

7. The retraction device 100 as claimed in claim 6, wherein the base plate 6 extends away from the first plate 1, to form a claw shape so as to engage with the retraction mounting system.

8. The retraction device 100 as claimed in claim 6, wherein the second plate 8 is connected to the base plate 6 through a pin joint to enable moveable connection.

9. The retraction device 100 as claimed in claim 6, wherein the first plate 1 and the second plate 8 comprises a clamping screw 11 mounted therebetween, and wherein the clamping screw 11 is mounted such that the clamping screw 11 is placed perpendicular to the first surface 2 and the second surface 7.

10. The retraction device 100 as claimed in claim 1, wherein the plurality of detachable hooks 9 are removably mounted on the first plate 1 via. a plurality of holes 3 on the first plate 1.

11. The retraction device 100 as claimed in claim 1, wherein the surgery is a brain surgery or a spine surgery or other surgery in humans.
